(19) United States
(12) Patent Application Publication
(10) Pub. No.: US 2005/0266077 A1
(43) Pub. Date: Dec. 1, 2005

(54) Resorbable matrices with coatings for delivery of bioactive compounds

(75) Inventor: Garfield P. Royer, Frederick, MD (US)

Correspondence Address: NIXON & VANDERHYE, PC, 901 NORTH GLEBE ROAD, 11TH FLOOR, ARLINGTON, VA, 22203, US

(73) Assignee: Royal Biomedical, Inc., Frederick, MD (US)

(21) Appl. No.: 10/518,035

(22) PCT Filed: Jun. 17, 2003
(86) PCT No.: PCT/US03/19006

§ 371 Date: Dec. 14, 2004

Related U.S. Application Data
(60) Provisional application No. 60/389,933, filed on Jun. 20, 2002.

(51) Int. Cl. A61K 9/26

(52) U.S. Cl. 424/469

(57) ABSTRACT

This invention relates to the production and use of coated inorganic-biopolymer complexes for the controlled release of bioactive compounds including medicinals. Advantageously, the delivery system compositions include an inorganic, a matrix polymer, and a coating. Advantageously, the inorganic used is calcium sulfate.

### FIELD OF THE INVENTION

This invention relates generally to the production and use of inorganic-polymer matrices with coatings. The matrices and coatings are resorbable. Sustained and/or controlled release of medicinal agents and other bioactive substances are the primary uses of these systems.

### BACKGROUND OF THE INVENTION

Plaster of Paris (POP) has been used without matrix biopolymers or medicinal complexing agents as CaSO₄-1/2H₂O [D. Mackey, et al, Clin. Orthop., 167, 263 (1982); and G. W. Bowyer, et al, J. Trauma, 36, 331 (1994)]. Polymethylmethacrylate and POP have been compared with regard to release profiles. Release rates from POP tend to be very fast.

Both polymethylmethacrylate and POP can be used to produce dimensionally stable beads and other structures. The acrylate cements or beads are formed by mixing pre-formed polymethylmethacrylate polymer, methylmethacrylate monomer, and a free-radical initiator.

An exothermic reaction ensues which results in matrix temperatures as high as 100° C. Many antibiotics such as polymyxin and tetracycline are inactivated by these conditions [G. J. Popham, et al, Orth. Rev., 20, 331 (1991)]. As mentioned above, polymethylmethacrylate is biocompatible but not resorbable. Therefore, beads used to treat local infection must be retrieved by surgery which is accompanied by the risk of re-infection. POP beads or pellets are resorbable but show inferior drug release profiles [G. W. Bowyer, et al, J. Trauma, 36, 331 (1994)].

Polymer matrices designed for controlled release of bioactive compounds can be non-resorbable or resorbable. In general, resorbable means degradable in the body by erosion from the surface or breakdown from within. The mechanism can involve either a chemical reaction, such as hydrolysis, or dissolution.

Non-resorbable polymers, such as polymethylmethacrylate, have been used for antibiotic delivery. These materials suffer from the disadvantage that they must be retrieved, which involves a second intervention and entails the risk of infection (H W Bucholz, et al., (1970) Chiburg, 43, 446).

Resorbable polymer matrices for controlled release are usually based on an oxygen-containing monomer, which is condensed in organic solvent to yield the polymeric product. The bioactive agent and the polymer are then combined in such a way as to give a timed-release formulation. The combination of active ingredient and polymer often involves organic solvents as well. The use of organic solvents is a decided disadvantage, especially when large-scale production is required. Toxic residues of organic solvents are a concern. Proteins and many polypeptides are incompatible with organic solvents.

The types of polymers in this category include:
polyesters
polyanhydrides
polyketals
poly(orthoesters)
polyurethanes
   (Burkersroda, F V and Goepferich, A M in Biomedical Materials, T Neenan, M Marcolongo and R F Valentini, eds. (1999), page 23, Materials Research Society, Warrendale Pa.).

Naturally occurring proteins may be used as structural components in drug-delivery matrices (Royer, U.S. Pat. No. 4,349,530; Royer, U.S. Pat. No. 5,783,214; Lee, Science (1981) 233-235). One deficiency of proteinaceous delivery matrices is that they can exhibit instability especially in environments where an inflammatory reaction is present such as a site of localized sepsis.

Commonly owned WO 99/15150 and U.S. Pat. No. 6,391,336 disclose stable, yet practical compositions for use in inflamed sites comprising an inorganic compound, a matrix polymer and/or a complexing agent. This composition has the advantage of being biocompatible but, unlike synthetic organic polymers, no non-aqueous solvents are required in the preparation. The drug is incorporated as a solid or as part of the matrix polymer solution. The material can also be used as a cement, that is, it can be injected directly into a lesion and allowed to solidify in situ.

Commonly owned U.S. Ser. No. 09/703,710 discloses a delivery system with a conditioning agent.

### OBJECTS OF THE INVENTION

It is an object of this invention to provide a safe resorbable delivery system that can be designed and fashioned to provide controlled release of bioactive substances over a pre-determined time-course.

It is an object of this invention to improve control of medicinal release rate and residence time.

### SUMMARY OF THE INVENTION

The subject invention relates to compositions for the controlled release of an active agent comprising an active agent and a matrix polymer dispersed throughout a matrix having a coating wherein said matrix is the hydration reaction product of an aqueous mixture comprised of:
an inorganic compound capable of undergoing hydration and/or crystallization, and
a matrix polymer,
wherein the inorganic compound of the matrix becomes a solid by hydration and/or crystallization.

Included within the invention are methods of producing the compositions and methods of producing sustained release of medicinals in mammals by administering the delivery systems with medicinals to mammals.

### DETAILED DESCRIPTION OF THE INVENTION

Introduction

The subject invention relates to a resorbable matrix with advantageous, i.e. sustained or controlled, release kinetics. The matrices are capable of releasing an active agent for a few days, e.g., 1, 2 or 3 days, 1, 2, or 3 weeks, or as many as 6 weeks. Inorganic compounds such as CaSO₄-1/2H₂O (calcium sulfate hemihydrate) can be combined with biopolymer in the presence of a bioactive agent including medicinals to produce a matrix, which is subsequently coated. Optionally, included are a complexing agent and a conditioning agent.

As used herein, the term “matrix polymer” refers to a polymer (often a biopolymer), which serves to control the erosion rate, setting time, and influences the release profile by raising the viscosity of the medium in the pores and channels of the delivery system. A “biopolymer” is defined as a pharmacologically acceptable polymer of biological or synthetic origin.

As used herein, the term “complexing agent,” refers to an agent (often a biopolymer), which is used to form a salt or conjugate with the active agent, which in effect raises the molecular weight of the active agent and lowers its rate of efflux. The complexing agent is typically a small molecule, which has affinity for the active agent. Pharmacologically acceptable hydrophobic medicinal complexing agents include proteins such as albumin, lipids or cyclodextrins, which can be used to complex neutral medicinal molecules or charged molecules, which contain a hydrophobic moiety. Liposomes containing a medicinal can be entrapped within the calcium sulfate matrix.

The delivery system of the subject invention for use with medicinals must meet the following requirements:
1. Safety—non-toxic, non-immunogenic, non-pyrogenic, non-allergenic.
2. Resorbablility—all components should be either assimilable or readily excreted.
3. Stability—the matrix should be sterilizable and precursors should have an acceptable shelf life. Cast forms should be dimensionally stable.
4. Compatibility—the materials and the preparative conditions should not alter the chemistry or activity of the medicinal.
5. Programmability—the residence time and release profile should be adjustable.

The inorganic compound-conditioning agent composites described herein are resorbable by dissolution. No acid is produced as opposed to hydrolytic erosion of polymer matrices such as polyesters.

Entrapment of bioactive substances within the resorbable biocompatible matrix described herein yields a delivery system, which permits controlled and localized release of a bioactive agent. Inorganic compounds such as CaSO₄-1/2H₂O can be combined with a polymer in the presence of a bioactive agent to produce a solid, which constitutes a biocompatible and resorbable delivery matrix (See WO 99/15150 and U.S. Pat. No. 6,391,336 the entire contents of which are incorporated by reference herein). The matrix is then coated.

Matrix Production

The production of the delivery system can be illustrated as follows: 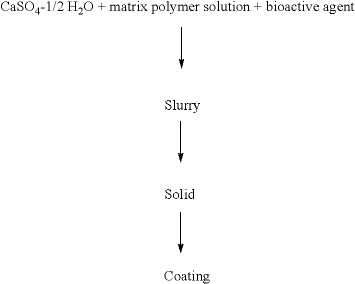
When contacted with water, calcium sulfate hemihydrate is converted to the dihydrate, CaSO, 2H₂O, which crystallizes. The mass of interlocking needle-like crystals produces a porous matrix with high compressive strength, as much as 2000 psi or more.

The slurry can be injected into molds to form spheres, cylinders, etc, or it can be allowed to solidify in bulk. In the latter case, the solid is milled and sized to yield microgranules. These microgranules can then be suspended in solution and injected. Microgranules can also be used in oral dosage forms.

A conditioning agent such as calcium stearate can be pre-mixed with the calcium sulfate hemihydrate. The slurry can be injected into the desired location with solidification in situ.

This composition is ideal for dental and orthopedic applications. The fact that the slurry can set-up in the presence of moisture is very advantageous.

The matrix is formed by the following reaction:

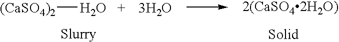

Normally, 1 g of calcium sulfate hemihydrate is treated with 0.6 ml of aqueous solution containing the matrix polymer along with dissolved or dispersed drug. The drug can also be incorporated into the formulation as a solid, ground with the calcium sulfate hemihydrate.

This formulation produces a hard porous mass of interlocking spherulitic crystals.

The inorganic-biopolymer complex can be formed as spheres, granules, cylinders, tablets and beads (including microbeads) for injection or for use in capsules. The latter can be formed by dispersing the slurry into a rapidly stirring water-immiscible medium. The size of the beads can be determined by the amount and nature of the surfactant and the stirring rate. Milling and sieving to produce beads/granules is an alternative approach. For orthopedic and dental use the inorganic-biopolymer complex matrix can be molded and or carved into specific shapes to conform to voids in bone structures. Just prior to formation of the intractable solid, the material is plastic and can be conveniently shaped to fit openings of irregular geometry.

Production of Dosage Forms

A delivery matrix of the invention can be produced by:
a. blending of an inorganic such as calcium sulfate hemihydrate and a conditioning agent such as calcium stearate, both in powder form,
b. mixing with matrix polymer solution (the drug can be dissolved or suspended in the polymer solution),
c. solidification in a mold or in bulk, and
d. unmolding or preparing microgranules by milling and sizing.

The molds, made of stainless steel or Teflon, can be used to prepare cylinders or spheres (e.g., both 3 mm in diameter). The preparation of wafers is also possible. Microgranules can in turn be compressed into tablets with various binding agents to yield another dosage form.

Surface coating with an erodible substance will block pores and slow efflux of drug until the coating agent is hydrolyzed or dissolved. It is possible to produce delayed release. Other embodiments include 2, 3 or more coatings and coatings with varying concentrations of coating polymer.

The delivery system typically has the following components:

1. Inorganic Compounds

Calcium sulfate hemihydrate is an advantageous inorganic component. The hemihydrate takes up water and crystallizes as the higher hydrate. Unadulterated calcium sulfate matrix exhibits poor drug release profiles. With conditioning agents, and optionally matrix polymers and complexing agent-active agent complexes the release profiles are improved. Other inorganics can be employed such as calcium silicates, aluminates, hydroxides and/or phosphates (see pages 72, 95, 327 in Reference Book of Inorganic Chemistry (1951) Latimer, W. H., and Hildebrand, J. M., Macmillan, New York, hereby incorporated by reference in its entirety).

2. Matrix Polymers

The preferred matrix polymers for medical use are biocompatible (non-toxic, non-allergenic, non-immunogenic), water soluble, and compatible with other components in the formulation.

Examples of matrix polymers include chondroitin sulfate, dextran (1-50%), hyaluronic acid (e.g., 1-5%), dextran sulfate, pentosan polysulfate, polyethylene glycol (PEG), polyvinylpyrrolidone (PVP), proteins such as collagen (gelatin), and fibrinogen. In an advantageous embodiment, a crosslinking agent is added to the matrix polymer. The addition of the crosslinking agent causes a reaction which leads to a higher molecular weight matrix polymer which increases viscosity in the pores. Diffusion is thereby inhibited. See Royer U.S. Pat. No. 6,391,336 and WO 99/15150, each being hereby incorporated by reference in its entirety. Counterions, are advantageously sodium or calcium. Chitosan as well as cationic polypeptides, polylysine, and polyarginine are examples of useful polymers that are positively charged at neutral pH.

The function of the matrix polymer is to control the viscosity, which is dependent on the nature, molecular weight and concentration of the polymer. The rationale for using polymers and polymeric complexing agents is based on Stokes law:
D is proportional to 1/Mv
D=the diffusion coefficient
M=the molecular weight of the medicinal
v=the viscosity of the medium
   3. Conditioning Agents

Conditioning agents are used to slow the erosion rate and permit solidification in the presence of moisture (repels water). Commonly owned U.S. Ser. No. 09/703,710, hereby incorporated by reference, discloses delivery systems with a conditioning agent.

All conditioning agents have a hydrophobic moiety. Calcium stearate is an advantageous choice for a conditioning agent that meets the criteria of safety and efficacy. Other calcium salts are useful in this regard. Examples include saturated and unsaturated carboxylic acids, aromatic carboxylic acids, corresponding phosphates, phosphonates, sulfates, sulfonates, and other compounds containing a hydrophobic moiety with a negatively charged anion. Salts of undecylenic acid are useful, in that they provide stability and also antifungal action. The use of calcium as the cation is advantageous but other cations will suffice; the group includes, but is not limited to, zinc, magnesium, aluminum and manganese.

The generalized chemical structure can be illustrated as follows:
R—X-M
where R is alkyl, alkenyl, alkynyl or aryl,
where X is a carboxylate, a carboxylic acid, an aromatic carboxylic acid, a corresponding phosphate, a phosphonate, a sulfate, or a sulfonate, and
where M is a metal ion such as calcium, zinc, magnesium, aluminum or manganese.

An example is calcium stearate, (CH₃ [CH₂]₁₆COO—)₂Ca²⁺

In this case R═CH₃[CH₂]₁₆, X═COO—, and M is the metal ion Ca²⁺. Cationic conditioning agents can also be employed, i.e.,
R—P—Y
where R=alkyl, alkenyl, alkynyl or aryl, where P=ammonium, or alkyl ammonium, and where Y=sulfate or phosphate.
4. Complexing Agents

To the extent that polymeric complexing agents increase the effective molecular weight of the active ingredient, the rate of efflux is slowed according to D is proportional to 1/Mv.

Complexing agents can be polymers or small molecules. The agents can form ionic bridges or hydrophobic bonds with the molecule to be delivered. The complexes involving the bioactive agents can range from sparingly soluble to soluble. Disodium pamoate is a good example of a complexing agent that forms sparingly soluble adducts with cationic bioactive ingredients. Disodium methylene disalicylate is a similar molecule to disodium pamoate that performs the same function. Procaine and benzathin can be used to reduce the solubility and rate of efflux of anionic bioactive agents. Additional complexing agents are presented in WO 99/15150.

5. Coatings

Substances useful as coatings which extend residence time, include i) biodegradable poorly water soluble or water insoluble materials suitable for blocking channels such as fibrin, polylactic acid (PLA), poly(lactide-co-glycolide) (PLGA), polycaprolactone (PCL), water insoluble small molecules such as triphenylphosphate and sucrose octa-acetate and acyl glycerols such as glyceryl tristearate or ii) biodegradable viscous water soluble agents such as hyaluronic acid, dextran, dextran sulfate (>100,000 MW), hydroxypropyl methyl cellulose, USP (EPMC), chitosan, and chondroitin sulfate.

The rate of dissolution of the coating influences the release profile.

A. Fibrin Coating

In order to coat the matrix with fibrin, drug is entrapped as usual by mixing calcium sulfate-hemihydrate with matrix polymer solution and allowing the mixture to set. The product is unmolded or processed as usual to microgranules. Water in external pores/channels is removed by drying overnight at room temperature.

The microbeads are wetted with fibrinogen solution (10% in Hepes buffer/30 mM, pH 7.2). The ratio of liquid to solid is balanced so that no excess solution exists in this particular example. When the solution volume exceeds the solid volume, the beads are dried to a “damp” state by removing excess polymer solution. This step can be done on a sintered glass filter under reduced pressure. Beads tend to stick together and are remilled to get a microbead preparation with the normal consistency.

The number of coating layers allows for control over the release profile. In the body fibrinogen is converted to fibrin. The stability of the fibrin layer can be adjusted by added fibrinoligase, the naturally occurring enzyme that catalyzes the cross-linking of fibrin clots.

Also, the inclusion of fibrinolysis inhibitors such as aprotinin and e-aminocaproate will slow down the degradation of the coating in vivo.

In another embodiment, the fibrinogen coating solution is diluted with water or another protein such as collagen or gelatin to change the effect of the coating.

The use of multiple coating layers and different additives allows preparation of a series of batches with different release profiles. The combination of fast-release, medium-release, and slow-release versions in varying proportions gives a resultant release profile, which can be tailored to the therapeutic requirement. It is possible to generate very close to a zero-order release. A final burst can also be obtained.

B. Organic Polymer Coating

1. Microgranules

This process of coating matrices for delivery of protein and non-protein active agents involves the following steps:
removing water from the matrix,
soaking of the matrix with polymeric coating solution-polymer in non-aqueous water miscible solvent such as NMP (N-methyl 2-pyrrolidinone), DMF (dimethylformamide) or THF (tetrahydrofuran),
removing trapped air, typically under reduced pressure,
solvent evaporation or exchange.

Optionally, the second step is to pretreat the porous matrix with solvent prior to soaking the matrix to enhance penetration by the coating solution. In some instances multiple coatings are desirable.

The use of multiple coating layers and different additives such as polysorb 80 or a second coating agent e.g. glyceryl tristearate allows preparation of a series of batches with different release profiles. The combination of fast-release, medium-release, and slow-release versions in varying proportions gives a resultant release profile, which can be tailored to the therapeutic requirement. Near zero-order release can be obtained.

In another embodiment, the polymeric coating solution contains drug, which provides additional loading.

The nature and amount of matrix polymer, the relative proportions of calcium sulfate hemihydrate and liquid, the complexing agent, and the nature and amount of the conditioning agent permit the adjustment of the release profile and residence time of the matrix.

2. Films/Fibers Containing Microgranules.

Homogeneous dispersions of matrix microgranules in a coating polymer can be spread onto glass plates to form films. These films can be useful for topical and transdermal drug delivery.

Use of NMP (N-methyl 2-pyrrolidinone)-microgranule-PLA mixtures can be used to make films of varying thickness. Injection into CaCl₂ solution will also yield “string” or fiber containing matrix microgranules. The characteristics of these fibers are dependent on the concentration of organic polymer, the medium into which it is injected and the stirring rate.

C. Matrix Beads Dispersed in Organic Polymers

In another embodiment, the matrix beads (or other shapes) are dispersed in the coating material (optionally including the active agent), and formed into cylinders and other various shapes.

Where the coating is a polymer with a melting point 40 C or above such as polycaprolactone (PCL), then a non-ionic surfactant such as polyoxyethylenesorbitan monooleate, (Tween 80, Polysorb 80, Span 80, Brij) can be added. The non-ionic surfactant can be adjusted as a means to regulate the release rate. This is primarily useful for delivery of non-protein active agents. This form of the matrix is typically made into cylinders, which can be made by molding or extrusion.

In this embodiment, matrix microgranules are typically mixed with molten organic polymer melt at >60 C and cooled to yield various shapes. The organic polymer is typically water insoluble. Cylinders are an advantageous form as they can be easily prepared and cut to size.

Polycaprolactone is an example of a bioerodible polymer that is useful in this application. Other examples are compounds with a melting point of 40 C and above. As above, free drug as well as drug formulated in microgranules can be employed in the dosage form. Additives such as Polysorbate 80 are included to influence the erosion rate and the release rate.

A representative formulation of a coated matrix follows:

| Ingredient | Amount | |
|---|---|---|
| Calcium sulfate hemihydrate | 1 | g |
| Drug | 50 | mg |
| Matrix polymer solution (10% w/v) | 0.6 | ml |
| Calcium stearate | 0.1 | g |
| Polylactic acid | 200 | mg |

When the amount of calcium sulfate hemihydrate is set at about 1 g, the amount of bioactive substance is set in the range of 1-300 mg and the matrix biopolymer in the range of 0.4-1 ml.

The concentration of the matrix polymer ranges from 0.1-50% (w/v). The conditioning agent is present in the range of 5-30% (w/w) based on calcium sulfate. The ratio of liquid/solid is advantageously 0.6.

The calcium sulfate hemihydrate can be sterilized by dry heat (140 for 4 hr); the polymer solution is sterilizable by filtration (0.2-micron filter). Terminal sterilization by gamma irradiation at 15-18 kGy is also effective.

A compilation of useful formulations is shown below in Table 1.

TABLE 1
Representative Coated Dosage Forms

| | Active | |
|---|---|---|
| Dosage Form | Ingredient | Polymer Coating |
| Microgranules | IgG | Hyaluronic Acid |
| Microgranules | IgG | HPMC |
| Microgranules | Growth hormone | PLGA |
| Microgranules | Growth hormone | Chitosan |
| Microgranules | Bupivacaine | PLA |
| Microgranules | Doxycycline | PLA |
| Cylinders | Doxycycline | PCL |
| Cylinders | Doxycycline | PCL/PS80 |
| Cylinders | Gentamicin | PCL |
| Cylinders | Gentamicin/pamoate | PCL |
| Cylinders | Bupivacaine | PCL |
| Cylinders | Bupivacaine | PCL/PS80 |
| Film | Silver sulfadiazine | PLA |
| Film | Silver sulfadiazine | HPMC |
| Film | Doxycycline | PLA |
| Fibers | Doxycycline | PLA |

HPMC = hydroxypropyl methyl cellulose, USP
NMP = N-methyl 2-pyrrolidinone
PCL = polycaprolactone, MW 10,000
PLA = poly (DL-lactic acid), MW 20,000
PLGA = poly (L-lactide co-glycolide) 70:30, Polyscience # 16587

Uses of the Matrix Compositions of the Invention

Medicinals (both non-protein drugs and medicinal proteins) useful with the matrices of the invention are presented in commonly owned WO 99/15150 and U.S. Ser. No. 09/703,710 each of which is hereby incorporated by reference. Therapeutics, antigens, antibodies including monoclonal antibodies, adjuvants, and regulatory molecules such as hormones exemplify bioactive agents with medical applications.

Various anti-infectives useful in conjunction with the formulations of the invention include gentamicin, clarithromycin, doxycycline, minocycline and lincomycin, amikacin, penicillin, cefazolin, ciprofloxacin, enrofloxacin, norfloxacin, silver sulfadiazine, imipenem, piperacillin, nafcillin, cephalexin, cefoperazone, vancomycin, tobramycin, nystatin, and amphotericin B or salts thereof (e.g., pamoate salt). Forming the pamoate (a complexing agent) of these anti-infectives to form complexes such as amikacin pamoate, clindamycin and gentamicin pamoate, are useful alone or in the formulations of the invention.

Cisplatin, paclitaxel, 5-FU, doxorubicin and other anti-neoplastic agents, can be delivered locally with beads (e.g., 3 mm) or with microgranules prepared as described herein. In one embodiment, localized administration is beneficial in that systemic toxicity is eliminated but concentrations in the area of cancerous tissue are high.

Vaccine antigens can be delivered with the system of the invention, for example, with microgranules (i.m. injection). The system of the invention can also be used to deliver DNA and RNA antigens.

The delivery system of the invention can also be used to deliver non-medical bioactive agents include sterilants, pheromones, herbicides, pesticides, insecticides, fungicides, algicides, growth regulators, antiparasitics, repellents, and nutrients. (See also WO 99/15150).

Modes of Administration

Administration of the solid matrix can be by surgical implant, oral, i.p., i.a. or p.a. The liquid injection can be s.c., i.m., or i.p. Advantageously, the administration is done by parenteral injection.

1. Slurry

1 g of calcium sulfate/calcium stearate (1-25% w/w) plus amikacin pamoate (100-320 mg) are thoroughly mixed and contacted with 0.6 ml of aqueous dextran sulfate (10% w/v).

After blending to a smooth slurry (30 s), the material is transferred to a 5 ml syringe and installed in vivo where it solidifies. Amikacin sulfate can be blended with amikacin pamoate to adjust the release profile. Presence of the calcium stearate allows for the solidification in the presence of moisture.

2. Beads/Cylinders

Sterile 3 mm beads can be installed individually with mosquito forceps or in groups using a cannula. A teat cannula is a safe tool for installation of beads and cylinders. This approach has been successfully used in the treatment of squamous cell carcinoma via intralesional chemotherapy with 3 mm beads of the invention containing cisplatin (7%).

3. Microgranules

a. Injection-Sterile microgranules (45-150 microns) (dry) are suspended in a suitable liquid for injection just prior to use. When antibiotics are involved, a solution of the antibiotic of choice may be used as the suspending liquid. For example, in treating a septic joint, amikacin solution (3 ml/25%) is used to suspend microbeads (300 mg) containing amikacin pamoate. An “initial burst” provided by the soluble amikacin sulfate is followed by the amikacin that elutes from the microbeads. A similar approach is appropriate for creating a subcutaneous depot of antibiotics and other active ingredients.
b. Oral-Microgranules are mixed with food or feed. The composition of the invention is tasteless and in some cases will mask the taste of a bioactive compound. In addition, the microgranules of the invention can be included in a capsule for oral delivery.

The following Examples are illustrative, but not limiting of the compositions and methods of the present invention. Other suitable modifications and adaptations of a variety of conditions and parameters normally encountered which are obvious to those skilled in the art are within the spirit and scope of this invention.

### EXAMPLES

Matrix Microgranule Formulations of the Examples

| Matrix | | | |
|---|---|---|---|
| Formulation | Matrix Polymer | CsCast + | Active Ingredient |
| I. Azoalbumin | 600 ul PEG (5%) | 1 g | 100 mg azoalbumin |
| II. IgG | 400 ul PEG (5%) | 670 mg | 34 mg IgG |
| | 40 mg DS500 | | (monoclonal |
| | | | antibody) |
| III. Lysozyme | 600 ul PEG (5%) | 1 g | 10 mg lysozyme |
| IV. Doxycycline | 600 ul PEG (10%) | 1 g | 160 mg |
| | | | doxycycline-HCL |
| V. Somatotropin | 600 ul PEG (5%) | 1 g | 300 mg |
| | | | somatotropin |

Abbreviations Used
CsCast=calcium sulfate/calcium stearate (95/5,wt/wt)
PBS=phosphate buffered saline (10 mM phosphate buffer-pH 7.4, 2.7 mM KCl, 13.7 mM NaCl)
PEG=polyethyleneglycol, MW 8,000
DS500=dextran sulfate MW 500,000
HPMC=hydroxypropyl methyl cellulose, USP
NMP=N-methyl 2-pyrrolidinone
PCL=polycaprolactone, MW 10,000
PLA=poly (DL-lactic acid), MW 20,000
PLGA=poly (L-lactide co-glycolide) 70:30, Polyscience # 16587

### Example 1

### Coating of Matrix-Azoalbumin (I) Microgranules with Hydroxypropyl Methyl Cellulose (HPMC)

300 mg of azoalbumin microgranules (I) was mixed with 600 mg of 5% HPMC (aq) to obtain a smooth suspension. The product was allowed to dry at room temperature for 24 hr with protection from light and dust. The dry material was milled and resized to 45-150 microns.

Release Profile

50 mg of coated beads was placed in a 2 ml centrifuge tube and overlayed with 500 μl PBS.

This was incubated at 37 C for 24 hrs and then centrifuged at 13,000 RPM for 5 minutes. The supernatant was removed and analyzed spectrophotometrically (450 nm). The process was repeated at 24 hr intervals for 4 days. The amount of protein in the eluent was calculated from a standard curve.

Release profile (1)

| Day | % Released |
|---|---|
| 1 | 14.5 |
| 2 | 3.6 |
| 3 | 3.4 |
| 4 | 4.1 |

### Example 2

### Coating of Matrix-Azoalbumin (I) Microgranules with Sucrose Octa-acetate

300 mg Matrix-Azoalbumin (I) microgranules was mixed with 200 μl of sucrose octa-acetate wt/vol in NMP) until all beads were wet.

This was left to dry at room temperature for 24 hours and protected from light and dust. The dried material was then milled and resized to obtain particles 45-150 microns.

Release Profile

50 mg of coated beads was placed in a 2 ml centrifuge tube with 500 μl PBS. This was incubated at 37 C for 24 hrs and then centrifuged at 13,000 RPM for 5 minutes. The supernatant was then analyzed spectrophotometrically (450 nm). The process was repeated at 24 hr intervals for 6 days. The amount of protein in the eluate was calculated from a standard curve.

Release profile (2)

| Day | % Released |
|---|---|
| 1 | 1.0 |
| 2 | 0.2 |
| 3 | 0.1 |
| 4 | 2.1 |
| 5 | 7.8 |
| 6 | 6.2 |

### Example 3

### Coating of Matrix-IgG (II) Microgranules with Fibrin

150 mg of Matrix-IgG (II) was mixed with 150 μl of 1% fibrinogen solution (porcine fibrinogen in 30 mM Hepes buffer pH 7.2) to obtain a smooth suspension. This material was then used directly or lyophilized.

Release Profile

The suspension was transferred into a 1 ml syringe and 501 injected into a 2-ml centrifuge tube. 500 μl PBS was added and the material incubated at 37 C for 24 hrs and then centrifuged at 13,000 RPM for 5 minutes. The supernatant was removed and analyzed spectrophotometrically (280 nm). Eluent from 50 μl blank beads (150 mg Matrix-beads (670 mg 5% CSCast, 40 mg dextran sulfate M.W. 500,000) was mixed with 150 μl of 1% fibrinogen solution) and was used as a control to compensate for background absorbance. The supernatant was removed and analyzed at 24 hr intervals for 4 days. The amount of released protein was calculated from a standard curve (A280).

Release profile (3)

| Day | % Released |
|---|---|
| 1 | 25.9 |
| 2 | 6.5 |
| 3 | 3.8 |
| 4 | 4.5 |

### Example 4

### Coating of Matrix-IgG (II) Microgranules with Hyaluronic Acid

150 mg Matrix-IgG (II) microgranules was mixed with 150 mg of 3% hyaluronic acid solution to obtain a smooth suspension. The suspension was injected directly or lyophilized as before.

Release Profile

The suspension was transferred into a 1 ml syringe and 50 μl injected to a 2 ml centrifuge tube. 500 μl PBS was added and the material was incubated at 37 C for 24 hrs. It was then centrifuged at 13,000 RPM for 5 minutes. The supernatant was removed and analyzed spectrophotometrically (280 nm). The process was repeated at 24 hr intervals for 6 days. The amount of protein released was calculated from a standard curve (A280).

Release profile (4)

| Day | % Released |
|---|---|
| 1 | 12.3 |
| 2 | 7.7 |
| 3 | 6.4 |
| 4 | 4.0 |
| 5 | 3.4 |
| 6 | 4.4 |

### Example 5

### Coating of Matrix-Lysozyme (III) Microbeads with Poly (L-lactide Co-glycolide) PLGA

300 mg-Lysozyme (III) microgranules was mixed with 300 μl of PLGA solution (10% wt/vol in NMP). The beaker containing the wet beads was placed in a dessicator and a vacuum pulled for 5 minutes. The material (not more than 3 mm thick) was spread on a glass tray protected from light and dust and left to dry at room temperature for 48 hours. The dried material was milled and sized to obtain particles 45-150 microns.

Release Profile

50 mg coated microgranules was placed a 2 ml centrifuge tube and 500 μl PBS was added. This was incubated at 37 C for 24 hrs and then centrifuged at 13,000 RPM for 5 minutes. The supernatant was removed; and analyzed spectrophotometrically (280 nm). The process was repeated at 24 hr intervals for 4 days. The amount of released protein was calculated from a standard curve (A280).

Release profile (5)

| Day | % Released |
|---|---|
| 1 | 18.5 |
| 2 | 5.1 |
| 3 | 4.5 |
| 4 | 3.3 |

### Example 6

### Coating of Matrix-Azoalbumin (I) Microgranules with Fibrin

300 mg Matrix-azoalbumin (I) microgranules was mixed with 200 μl of 10% fibrinogen solution (porcine fibrinogen in 30 mM Hepes buffer pH 7.2) and left to dry at room temperature for 24 hours while being protected from light and dust. The material was not sealed. It was then milled and sized to obtain particles of 45-150 microns.

Release Profile

100 mg of coated microgranules was placed in 2 ml centrifuge tube and 900 μl PBS plus 100 μl thrombin solution (4.7 units/ml bovine thrombin in 30 mM Hepes pH7.2, 15N NaCl and 25% Glycerol) was added. This was incubated at 37 C for 24 hrs and then centrifuged at 13,000 RPM for 5 minutes. The supernatant was removed from the centrifuge tube; and analyzed spectrophotometrically (450 nm). The process was repeated at 24 hr intervals for 4 days. The amount of released protein was calculated from a standard curve.

Release profile (6)

| Day | % Released |
|---|---|
| 1 | 1.4 |
| 2 | 1.9 |
| 3 | 1.4 |
| 4 | 1.0 |

### Example 7

### Cylinders Containing Matrix Doxycycline (IV) Microgranules with Polycaprolactone

1 g PCL (Ave. M.W. 10,000) was placed into a 25 ml beaker and warmed to 75 C for 30 minutes or until melted. The temperature was reduced to 65 C and 1 g of matrix doxycycline microgranules (45-150μ) was added; the material was mixed to form a smooth slurry. The material was transferred to a 3 ml syringe with the aid of a spatula. The syringe was warmed to 65 C and the contents were injected into a cylindrical mold (ID=3 mm). After a setting time of at least 30 minutes, the cylinders were unmolded and cut to the desired length.

Release Profile

100 mg cylinder was placed in 2 ml centrifuge tube. 1 ml PBS was added and the sample was incubated at 37 C for 24 hrs. The supernatant was removed, centrifuged at 13,000 rpm for 5 minutes and analyzed spectrophotometrically (351 nm). The process was repeated at 24 hr intervals 4 days. The amount of released drug was calculated from a standard curve (A351).

Release profile (7)

| Day | % Released |
|---|---|
| 1 | 1.1 |
| 2 | 0.5 |
| 3 | 0.3 |
| 4 | 0.3 |

### Example 8

### Cylinders Containing Matrix Doxycycline (IV) Microgranules and Polycaprolactone(PCL)/Polysorbate 80

500 mg polycaprolactone (Ave. M.W. 10,000) was placed into a 25 ml beaker and warmed to 75 C for 30 minutes or until melted. 500 μl of Polysorbate 80 was added and the material stirred until homogeneous. The temperature was reduced to 65 C and 1 g of matrix doxycycline microgranules (45-150μ) was added and the material was mixed to form a smooth slurry. The material was transferred to a 3 ml syringe with the aid of a spatula. The syringe was warmed to 65 C and the contents injected into a cylindrical mold (ID=3 mm). The setting time was 15 minutes. The cylinders were unmolded and cut to the desired length.

Release Profile

100 mg cylinder was placed in a centrifuge tube. 1 ml PBS was added and the material was incubated at 37 C for 24 hrs. The supernatant was removed from the centrifuge tube, centrifuged at 13,000 RPM for 5 minutes; and analyzed spectrophotometrically (351 nm). The process was repeated at 24 hr intervals for 4 days. The amount of released drug was calculated from a standard curve (A351).

Release profile (8)

| Day | % Released |
|---|---|
| 1 | 12.7 |
| 2 | 6.0 |
| 3 | 3.3 |
| 4 | 2.6 |

Variation of the amount of Polysorbate 80 can be a useful tool in adjusting the release profile to meet the demands of the therapeutic situation. This point is illustrated as shown below:

| % PS80 | % Release, Day 1 |
|---|---|
| 25% | 7.6% |
| 50% | 13% |

### Example 9

### Cylinders Containing Matrix Doxycycline (IV) Microgranules and Polycaprolactone (PCL) Containing Doxycycline

1 g PCL was placed into a 25 ml beaker and the material was warmed to 75 C for 30 minutes or until melted. The temperature was reduced to 65 C and 1 g of Matrix Doxycycline microgranules (45-150μ) and 100 mg of Doxycycline-HCL was added; the material was then mixed to form a smooth slurry. The material was transferred to a 3 ml syringe with the aid of a spatula. The syringe was warmed to 65 C and the contents were injected into a cylindrical mold (ID=3 mm). The setting time was 30 minutes. The cylinders were unmolded and cut to the desired length.

Release Profile

A 100 mg cylinder was placed in a 2 ml centrifuge tube. 1 ml PBS was added and the material incubated for 37 C for 24 hrs. The supernatant was removed and analyzed spectrophotometrically (351 nm). The process was repeated at 24 hr intervals for 4 days. The amount of released drug was calculated from a standard curve (A351).

Release profile (9)

| Day | % Released |
|---|---|
| 1 | 2.3 |
| 2 | 0.8 |
| 3 | 0.6 |
| 4 | 0.5 |

### Example 10

### Coating of Doxycycline Microgranules (IV) with Poly(DL-lactic acid)PLA Containing Doxycycline

PLA was dissolved in NMP by warning at 60 C (2 g PLA with 2 ml NMP); and then allowed to cool to room temperature. Doxycycline was added to achieve a concentration of 10% (w/w). 1 g of the PLA/doxycycline solution was mixed with 1 g of doxycycline microgranules (IV) to obtain a homogeneous paste.

This paste can be used directly by forming into various shapes and installing at a surgical site such as a periodontal defect. The paste can be warmed and installed by injection. As an alternative the mixture can be injected into a rapidly stirring aqueous solution to give spherical beads, the size of which is dependent upon stirring rate and the presence of surfactants.

Another option is a “string” which can be kept as a coil and formed readily into the desired shape by the health care professional just prior to use. This dosage form is obtained by simply injecting the above mixture in unstirred water and coiling the “string” onto a glass rod.

Another alternative is to make semi-cylinders using a Teflon mold. The mold has open troughs in the form of semi-cylinders, which are milled such that the width at the top is 3 mm. The mold is filled with a syringe and the solvent is removed in vacuo until a dosage form of desired consistency is achieved.

### Example 11

### Films Containing Doxycycline Microgranules (IV) and Poly(DL-lactic acid)PLA

PLA-NMP solution was prepared (23% w/w). 100 mg Doxycycline microgranules (IV) were mixed with the PLA solution (200 μl) to give a smooth slurry. The mixture was spread onto a glass plate and allowed to air dry for 48 hrs while protected from light and dust.

### Example 12

### Coating of Matrix-Somatotropin (V) with Poly-DL-Lactide-Co-Glycolide (PLGA)

300 mg Matrix-Somatotropin (V) microgranules were placed into a 10 ml beaker. 300 μl of poly-DL-lactide-co-glycolide solution (5% wt/vol in 1-Methyl-2-pyrrolidinone) was added and the material was mixed until all beads were wet. The beaker containing the wet beads was placed in a dessicator and a vacuum pulled for 5 minutes or until no air bubbles were observed. The material was spread (not more than 3 mm thick) on a glass tray and left to dry at room temperature for 48 hours. The tray was covered lightly to protect from dust. It was not sealed. The dry material was milled using a mortar and pestle; and sized to obtain particles 45-150 microns.

50 mg coated beads were placed in a 2 ml centrifuge tube with 500 μl PBS buffer. This mixture was incubated in a water bath at 37° C. for 24 hrs. The supernatant was removed and then centrifuged 13,000 RPM for 5 minutes and analyzed spectrophotometrically (280 nm). The process was repeated at 24 hr intervals for 5 days. The amount of released protein was calculated from a standard curve (A280).

Release profile (12)

| Day | % Released |
|---|---|
| 1 | 4.6 |
| 2 | 0.4 |
| 3 | 1.2 |
| 4 | 2.0 |
| 5 | 2.2 |

It will be readily apparent to those skilled in the art that numerous modifications and additions may be made to the present invention, the disclosed device, and the related system without departing from the invention disclosed.

1. A composition for the controlled release of an active agent comprising an active agent and a matrix polymer dispersed throughout a matrix having a coating wherein said matrix is the hydration reaction product of an aqueous mixture comprised of:
an inorganic compound capable of undergoing hydration and/or crystallization, and
a matrix polymer,
wherein said inorganic compound of said matrix becomes a solid by hydration and/or crystallization.

2. A composition as in claim 1, wherein said inorganic compound is calcium sulfate hemihydrate.

3. A composition as in claim 1, wherein said matrix polymer is a biopolymer selected from the group consisting of hyaluronic acid, chondroitin sulfate, dextran, dextran sulfate, and polyethylene glycol.

4. A composition as in claim 3, wherein said matrix polymer is dextran sulfate.

5. A composition as in claim 3, wherein said matrix polymer is polyethylene glycol.

6. A composition as in claim 1, further comprising a conditioning agent.

7. A composition as in claim 6, wherein said conditioning agent is selected from the group consisting of calcium stearate, zinc undecylenate, magnesium palmitate, sodium laurate, calcium napthenate, calcium oleate, lauryl and ammonium sulfate.

8. A composition as in claim 6, wherein said conditioning agent is calcium stearate.

9. A composition as in claim 1, further comprising a complexing agent.

10. A composition as in claim 1, further comprising a complexing agent selected from the group consisting of chondroitin sulfate, polyglutamic acid, polyaspartic acid, pamoic acid, polynucleotides, a cationic polypeptide, cyclodextrin, polyoxyethylene alcohol, ester or ether, and defatted albumin.

11. A composition as in claim 1, wherein said coating is a biodegradable poorly water soluble or water insoluble agent suitable for blocking channels of said matrix.

12. A composition as in claim 11, wherein said coating is selected from the group consisting of fibrin, polylactic acid (PLA), poly(lactide-co-glycolide) (PLGA), and polycaprolactone (PCL).

13. A composition as in claim 1, wherein said coating is fibrin.

14. A composition as in claim 11, wherein said coating is selected from the group consisting of triphenylphosphate and sucrose octa-acetate and other acyl sugar derivatives, and acyl glycerols such as glyceryl tristearate.

15. A composition as in claim 1, wherein said coating is a biodegradable viscous water soluble agent suitable for blocking channels of said matrix.

16. A composition as in claim 15, wherein said coating is selected from the group consisting of hyaluronic acid, dextran, dextran sulfate (>100,000 MW), HPMC, chitosan, and chondroitin sulfate.

17. A composition as in claim 16, wherein said coating is dextran.

18. A composition as in claim 16, wherein said coating is HPMC.

19. A composition as in claim 1, wherein said system is in the form of a bead, a fiber, a wafer, a tablet, a sphere, a granule or a cylinder.

20. A composition as in claim 1, wherein said system is in the form of a cylinder and said matrix is dispersed in said coating.

21. A composition as in claim 20 wherein said coating is polycaprolactone (PCL).

22. A composition as in claim 21, further comprising a non-ionic surfactant in said coating.

23. A composition as in claim 21, further comprising active agent in said coating.

24. A composition as in claim 1, comprising calcium sulfate dihydrate, calcium stearate, glycosaminoglycan, and a coating.

25. A composition as in claim 24, wherein said glycosaminoglycan is hyaluronic acid or chondroitin sulfate.

26. A composition as in claim 1, comprising calcium sulfate dihydrate, calcium stearate and hyaluronic acid and fibrin.

27. A composition as in claim 1, wherein said active agent is a medicinal.

28. A composition as in claim 27, wherein said medicinal is a salt.

29. A composition as in claim 27, wherein said medicinal is a protein.

30. A composition as in claim 27, wherein said medicinal is a growth factor.

31. A composition as in claim 27, wherein said medicinal is a drug precursor.

32. A composition as in claim 27, wherein said medicinal is a cytokine or a colony stimulating factor.

33. A composition as in claim 27, wherein said medicinal is an anti-infective selected from the group consisting of gentarnicin, clarithromycin, doxycycline, minocycline and lincomycin, amikacin, penicillin, cefazolin, ciprofloxacin, enrofloxacin, norfloxacin, silver sulfadiazine, imipenem, piperacillin, nafcillin, cephalexin, cefoperazone, vancomycin, tobramycin, nystatin, silver sulfadiazine, imipenem, and amphotericin B or salts thereof.

34. A composition as in claim 27, wherein said medicinal is an antibiotic.

35. A composition as in claim 27, wherein said medicinal is an antineoplastic agent.

36. A composition as in claim 27, wherein said medicinal is an anesthetic.

37. A composition as in claim 1, wherein said active agent is a non-medicinal compound.

38. A composition as in claim 37, wherein said non-medicinal compound is selected from the group consisting of a sterilant, a pheromone, a herbicide, a pesticide, an insecticide, a fungicide, an algicide, a growth regulator, a nematicide, a repellent, and a nutrient.

39. A method of producing sustained release of a medicinal in a mammal comprising administering the composition of claim 1 wherein said active agent is a medicinal to said mammal.

40. A method as in claim 39, wherein said administration is by subcutaneous injection.

41. A method of treating an infection in a mammal comprising administering the composition of claim 1 wherein said active agent is an anti-infective to said mammal.

42. A method of producing a composition for the controlled release of an active agent comprising:
(a) mixing an active agent, an inorganic compound capable of undergoing hydration and/or crystallization, and a matrix biopolymer, and
(b) drying the product of step (a) and
(c) coating the product of step (b).

43. A method as in claim 42, wherein said inorganic compound, and a conditioning agent are premixed and then added to said matrix biopolymer.

44. A method as in claim 42, wherein step (c) comprises i) dispersing the product of step (b) into a molten polymer and ii) molding the product of i) into a predetermined shape.
